(19) United States
(12) Patent Application Publication
(10) Pub. No.: US 2018/0135138 A1
(43) Pub. Date: May 17, 2018

(54) METHOD FOR EVALUATING HEALTH CONDITION AND METHOD FOR PREDICTING LONG-TERM EFFICACY OF ANTICANCER AGENT

(71) Applicants: TOPPAN PRINTING CO., LTD., Tokyo (JP); NIPPON MEDICAL SCHOOL FOUNDATION, Tokyo (JP)

(72) Inventors: Shiro Kitano, Tokyo (JP); Takeshi Yamada, Tokyo (JP); Takuma Iwai, Tokyo (JP)

(73) Assignees: TOPPAN PRINTING CO., LTD., Tokyo (JP); NIPPON MEDICAL SCHOOL FOUNDATION, Tokyo (JP)

(21) Appl. No.: 15/870,190

(22) Filed: Jan. 12, 2018

Related U.S. Application Data
(63) Continuation of application No. PCT/JP2016/070963, filed on Jul. 15, 2016.

(30) Foreign Application Priority Data
Jul. 17, 2015 (JP) 2015-143300
Mar. 10, 2016 (JP) 2016-047523

(51) Int. Cl. C12Q 1/6886

(52) U.S. Cl. CPC C12Q 1/6886

(57) ABSTRACT

A method for evaluating a health condition, includes: measuring a test amount of cell free DNA per unit amount of a body fluid sample collected from a subject after tumor resection; and comparing the measured test amount of cell free DNA with a reference value so as to evaluate a health condition of the subject. The reference value is one of a predetermined threshold value; an amount of cell free DNA per unit amount of a body fluid sample collected from the subject before tumor resection; an amount of cell free DNA per unit amount of a body fluid sample collected from the subject between 1 week and 3 months after tumor resection; and an amount of cell free DNA per unit amount of a body fluid sample collected from the subject by the time of collecting the body fluid sample for measuring the test amount of cell free DNA.

### CROSS REFERENCE TO RELATED APPLICATIONS

This application is a continuation application based on a PCT Patent Application No. PCT/JP2016/070963, filed Jul. 15, 2016, whose priority is claimed on Japanese Patent Application No. 2015-143300, filed on Jul. 17, 2015, and Japanese Patent Application No. 2016-047523, filed Mar. 10, 2016, the entire contents of which are hereby incorporated by reference.

### BACKGROUND OF THE INVENTION

### Field of the Invention

The present invention relates to a method for evaluating a health condition, particularly tumor recurrence or metastasis of a cancer patient, particularly a cancer patient after tumor resection, and for evaluating efficacy, side effects, and the like of chemotherapy in a relatively noninvasive manner based on the amount of cell free DNA (cfDNA) in a body fluid.

### Description of the Related Art

Somatic mutation on oncogenes involved in signal transduction has attracted attention as a predictive factor for a therapeutic effect of an anticancer agent. Examples thereof include RAS gene mutation as a predictive factor for a therapeutic effect of cetuximab or panitumumab, which is an anti-EGFR (epidermal growth factor receptor) antibody drug that is a molecular-targeted drug in a therapy of colorectal cancer. In addition, there are point mutation, deletion, and the like of EGFR as a predictive factor for a therapeutic effect of a molecular-targeted drug of low molecular weight compounds such as gefitinib and erlotinib in a therapy of lung cancer. Furthermore, a BCR-ABL fusion gene as a predictive factor for imatinib efficacy in leukemia, an EML4-ALK fusion gene in an EGFR-negative lung cancer patient, and the like are also being examined at the actual clinical site.

In order to predict the therapeutic effect of a molecular therapeutic agent, somatic mutation of a tumor tissue is generally an indicator, and therefore a genotype of an oncogene in a tumor tissue sample collected by biopsy is examined. In addition, because somatic mutation may occur at any time, it is desirable to monitor the somatic mutation, which is a predictive factor for a therapeutic effect, over time, so as to understand the state of cancer in real time. However, most patients receiving chemotherapy have tumors in a deep parts of the body such as lung and liver. Therefore, biopsy from these sites is significantly invasive for a patient and it is difficult to perform biopsy over time.

On the other hand, in recent years, it has been reported that the amount of cell free (cf) DNA in blood is higher in cancer patients than in healthy subjects, and the amount of cfDNA in blood can be expected as a tumor marker (for example, refer to Schwarzenbach, et al., Nature Review, 2011, vol. 11, pp. 426 to 437). In addition, it has been reported that whether or not drug resistance of the anti-EGFR antibody drug is obtained with respect to colorectal cancer correlates with somatic mutation of a KRAS gene in cfDNA in blood as well as of somatic mutation of a KRAS gene in a tumor tissue. That is, it has been reported that the state of a tumor can be understood by detecting somatic mutation of a KRAS gene in cfDNA in blood (for example, refer to Misale, et al., Nature, 2012, vol. 486 (7404), pp. 532 to 536, and Diaz, et al., Nature, 2012, vol. 486 (7404), pp. 537 to 540). Even if it is difficult for a normal biopsy to repeat the biopsy, monitoring of somatic mutation is becoming possible by using blood as an alternative specimen of tissue.

However, in order to detect genetic mutation from cancer cells and the like in peripheral blood, it is necessary to collect large amounts of peripheral blood at once, which is a physical heavy burden for cancer patients. In addition, for detection of genetic mutation, it usually takes about two weeks and time is needed to obtain the results, which also leads to delays in on-site doctor's judgment for therapy. Therefore, information for predicting therapeutic effects which is available more quickly is required. It can be expected to reduce the burden on patients in clinical tests and to speed up the determination for therapy, because cfDNA can be easily recovered from a small amount of blood.

### SUMMARY

An object of the present invention is evaluating the health condition, particularly tumor recurrence, tumor metastasis, the efficacy, side effects, and the like of chemotherapy of a cancer patient, particularly a cancer patient after tumor resection, based on an amount of cfDNA in samples such as a body fluid collected in a less invasive manner than the collection of tissue by biopsy.

As a result of extensive research to solve the above problems, the inventors of the present invention have found that an amount of cfDNA in the body fluid increases in a short term by tumor resection treatment, but the amount stabilizes at a low level thereafter, and that the amount thereof tends to increase in a case where tumor recurrence or metastasis occurs or side effects caused by chemotherapy occur, and have completed the present invention.

A method for evaluating a health condition according to a first aspect of the present invention includes: measuring an test amount of cell free DNA per unit amount of a body fluid sample collected from a subject after tumor resection; and comparing the measured test amount of cell free DNA with a reference value so as to evaluate a health condition of the subject; wherein the reference value is any one of a predetermined threshold value; an amount of cell free DNA per unit amount of a body fluid sample collected from the subject before the tumor resection; an amount of cell free DNA per unit amount of a body fluid sample collected from the subject between 1 week and 3 months after the tumor resection; and an amount of cell free DNA per unit amount of a body fluid sample collected from the subject by the time of collecting the body fluid sample for measuring the test amount of cell free DNA, and when evaluating the health condition of the subject, in a case where the test amount of cell free DNA is higher than the reference value, it is evaluated that a tumor of the subject has increased or new metastasis has occurred or there is a high possibility of an increase of a tumor or of occurrence of new metastasis, or that while a tumor of the subject has not increased and no new metastasis has occurred, the health condition has deteriorated or there is a high possibility of deterioration of the health condition.

In the first aspect, the body fluid sample may be a body fluid sample collected from the subject after the tumor resection over time, and the test amount of cell free DNA per unit amount of the body fluid sample may be monitored so as to evaluate the health condition of the subject over time.

In the first aspect, in a case where the test amount of cell free DNA per unit amount of the body fluid sample of the subject is increasing, it may be evaluated that a tumor of the subject has increased or new metastasis has occurred or there is a high possibility of an increase of a tumor or occurrence of new metastasis, or while a tumor of the subject has not increased and no new metastasis has occurred, the health condition has deteriorated or there is a high possibility of deterioration of the health condition.

In the first aspect, the subject may have received chemotherapy, and when evaluating the health condition of the subject, in a case where the test amount of cell free DNA is higher than the reference value, it may be evaluated that a side effect due to the chemotherapy has occurred or there is a high possibility of occurrence of the side effect due to the chemotherapy.

In the first aspect, the subject may have received chemotherapy, and when evaluating the health condition of the subject, in a case where the test amount of cell free DNA is higher than the reference value, it may be evaluated that the chemotherapy is ineffective.

In the first aspect, a chemotherapeutic agent used for the chemotherapy may be one or more selected from the group consisting of fluorouracil, leucovorin, oxaliplatin, capecitabine, tegafur/gimeracil/oteracil potassium, irinotecan, bevacizumab, cetuximab, and panitumumab.

In the first aspect, the reference value may be an amount of cell free DNA per unit amount of the body fluid sample collected from the subject when 42 to 90 days have passed from the tumor resection.

In the first aspect, the body fluid sample may be selected from the group consisting of blood, serum, plasma, urine, saliva, semen, thoracic exudate, cerebrospinal fluid, tears, sputum, mucus, lymph fluid, cytosol, ascites, pleural effusion, amniotic fluid, bladder lavage solution, and bronchoalveolar lavage solution.

In the first aspect, the body fluid sample may be serum or plasma.

In the first aspect, the reference value may be 1000 ng of an amount of cell free DNA per mL of plasma.

In the first aspect, the amount of cell free DNA per unit amount of the body fluid sample may be measured by an absorbance method, an intercalation method, a real-time PCR method, a digital PCR method, a next generation sequencing method, or an electrochemical detection method.

In the first aspect, the tumor may be one of the group consisting of metastatic medulloblastoma, gastrointestinal stromal tumor, dermatofibrosarcoma protuberans, colon rectum cancer, colorectal cancer, lung cancer, non-small-cell lung cancer, small cell lung cancer, chronic myeloproliferative disease, acute myelogenous leukemia, thyroid cancer, pancreatic cancer, bladder cancer, kidney cancer, melanoma, breast cancer, prostate cancer, ovarian cancer, cervical cancer, head and neck cancer, brain tumor, hepatocellular carcinoma, hematologic malignancy, and pre-cancer causing these cancers.

In the first aspect, the evaluated health condition of the subject may used as a material for determining whether to continue or stop the chemotherapy or to change the chemotherapeutic agent to be used.

A method for predicting long-term efficacy of an anticancer agent according to a second aspect of the present invention, the method including: measuring an amount of cell free DNA per unit amount of a body fluid sample collected from a subject through two or more collections over time so as to monitor the amount of cell free DNA per unit amount of the body fluid sample of the subject; and comparing the obtained amount of cell free DNA with a predetermined reference value; wherein it is predicted that long-term efficacy of an anticancer agent is obtained for the subject in a case where the amount of cell free DNA decreases from a value greater than the reference value to the reference value or less, or in a case where the amount of cell free DNA of each body fluid sample obtained through all of the collections is equal to or less than the reference value; and it is predicted that long-term efficacy of the anticancer agent is not obtained for the subject in a case where the amount of cell free DNA increases from a state equal to or less than the reference value to a value greater than the reference value, or in a case where the amount of cell free DNA of each body fluid sample obtained through all of the collections is greater than the reference value.

In the second aspect, the body fluid sample may be serum or plasma.

In the second aspect, the reference value may be 20 ng of the amount of cell free DNA per mL of serum or plasma.

In the second aspect, the long-term efficacy may mean that a volume increase, metastasis, or recurrence of tumor tissue does not occur for at least 6 months.

In the second aspect, the subject may suffer from one or two or more selected from the group consisting of colorectal cancer, colon cancer, rectal cancer, lung cancer, liver cancer, breast cancer, ovarian cancer, prostate cancer, renal cancer, esophageal cancer, head and neck cancer, uterine cancer, and cervical cancer.

In the second aspect, the anticancer agent may be one or more selected from the group consisting of an EGFR inhibitor and a VEGF inhibitor.

According to the method for evaluating a health condition according to the above aspects of the present invention, the health condition, particularly tumor recurrence or metastasis, and the efficacy, side effects, and the like of chemotherapy of a subject after tumor resection, can be relatively noninvasively evaluated with high sensitivity without requiring an invasive and burdensome examination such as biopsy.

In addition, according to the method for predicting long-term efficacy of an anticancer agent according to the above aspects of the present invention, the long-term efficacy of the anticancer agent can be relatively noninvasively predicted with respect to a subject receiving therapy of the anticancer agent.

### BRIEF DESCRIPTION OF THE DRAWINGS

FIG. 1 is a diagram showing a dosage regimen of FOLFOX chemotherapy in Example 1.

FIG. 2 is a diagram showing a dosage regimen of a combination therapy of mFOLFOX+panitumumab in Example 1.

FIG. 3 is a diagram showing a dosage regimen of XELOX chemotherapy in combination with bevacizumab in Example 1.

FIG. 4 is a diagram showing a change in cfDNA amount of Patient A over time in Example 1.

FIG. 5 is a diagram showing a change in cfDNA amount of Patient B over time in Example 1.

FIG. 6 is a diagram showing a change in cfDNA amount in Patient C over time in Example 1.

FIG. 7 is a diagram showing a change in cfDNA amount of Patient D over time in Example 1.

FIG. 8 is a diagram showing a change in cfDNA amount of Patient E over time in Example 1.

FIG. 9 is a diagram showing a change in cfDNA amount of Patient F over time in Example 1.

FIG. 10 is a diagram showing a change in cfDNA amount of Patient G over time in Example 2.

FIG. 11 is a diagram showing a dosage regimen of XELOX chemotherapy in combination with bevacizumab in Example 3.

FIG. 12 is a CT imaging diagram of a cancer patient in Case 4 in Example 3.

FIG. 13 is a CT imaging diagram of a cancer patient in Case 5 in Example 3.

### DETAILED DESCRIPTION OF THE PREFERRED EMBODIMENTS

Method for Evaluating Health Condition

A method of evaluating a health condition according to an embodiment of the present invention is characterized by evaluating a health condition of a subject after tumor resection by using an amount of cfDNA in a body fluid as an indicator. Specifically, the method includes a quantification step of measuring a test amount of cfDNA per unit amount of a body fluid sample collected from a subject after tumor resection, and an evaluation step of evaluating a health condition of the subject by comparing the test amount of cfDNA obtained in the quantification step with a reference value.

As shown in examples described later, the amount of cfDNA per unit amount in a body fluid such as peripheral blood of a cancer patient increases as the malignancy of the tumor increases. For example, the amount of cfDNA per unit amount in a body fluid increases as a volume of tumor tissue increases. If metastasis occurs, the amount thereof increases more than before the metastasis, and if recurrence occurs, the amount thereof becomes more than before the recurrence. Therefore, the amount of cfDNA per unit amount in a body fluid can be used as an indicator for evaluating the possibility of metastasis or recurrence, and the like.

In addition, in a case where a tumor patient is receiving therapy such as chemotherapy and the therapy is appropriately effective, a tumor becoming malignant (increase of the tumor or new metastasis) is suppressed in the patient. Therefore, the amount of cfDNA per unit amount in a body fluid does not increase so much. On the other hand, in a case where the therapy is not appropriately effective, tumor growth and the like progresses in the patient. As a result, the amount of cfDNA per unit amount in a body fluid increases. That is, the amount of cfDNA per unit amount in a body fluid can be used as an indicator for evaluating or predicting the efficacy of therapy such as chemotherapy.

In a case where a tumor patient is receiving chemotherapy in which relatively severe side effects are problematic, and even in a case where the chemotherapy is effective and tumor growth and the like is suppressed, if the health condition of the patient deteriorates due to the side effects, there is a tendency that the amount of cfDNA per unit amount in a body fluid such as peripheral blood increases. In fact, the amount of DNA in a patient in whom other disease complications such as pneumonia are caused also tends to increase due to side effects of chemotherapy. Therefore, the amount of cfDNA per unit amount in a body fluid can also be used as an indicator for evaluating the presence or absence of side effects of chemotherapy and the like or the intensity of side effects thereof, or for predicting the risk of side effects thereof.

As above, a health condition of a cancer patient after tumor resection, for example, recurrence and a possibility thereof, metastasis and a possibility thereof, a drug efficacy state, side effects, and the like, can be evaluated based on the amount of cfDNA per unit amount in a body fluid. In the related art, it has been known that the amount of cfDNA in peripheral blood of a cancer patient increases compared to a healthy subject. However, it has not yet been reported that there is a correlation between this knowledge of the related art and a drug efficacy state, a possibility of metastasis, a possibility of recurrence, a state of side effects, and the like.

In a cancer patient after tumor resection, it is important to quickly detect and receive appropriate therapy in a case where recurrence or metastasis occurs. Therefore, the presence or absence of recurrence or metastasis is periodically examined. In this case, from the viewpoint of invasiveness to a subject, costs, and the like. CT (computed tomography) is not frequently used and a method for monitoring very common tumor markers such as CEA and CA-19 in peripheral blood (blood) is used for clinical practice (according to Japanese National Health Insurance, CT is approved once every 6 months and blood sampling is approved once a month). However, it is not unusual for these tumor markers not to show abnormal values even if tumors are presented. In addition, even if a subject is a healthy subject or is a patient with a tumor suffers from a disease other than the tumor, a value thereof often happens to exceed a reference value and there is a problem in that the accuracy is low. In the method for evaluating a health condition according to the present embodiment, a cancer patient after tumor resection is evaluated based on the amount of cfDNA per unit amount in a body fluid. Therefore, a highly reliable evaluation with a relatively high degree of accuracy can be expected compared to cases based only on specific tumor markers such as CEA and CA-19.

Furthermore, as a method for predicting a therapeutic effect, for example, KRAS gene mutation is used as a predictive factor for a therapeutic effect of an EGFR inhibitor. However, there are cases in which the EGFR inhibitor do not exhibit the efficacy even in a patient not having KRAS gene mutation. In addition, in a case of monitoring postoperative recurrence, it is limited to a case of a patient whose primary tumor was KRAS gene mutant. On the other hand, in the method for evaluating a health condition according to the present embodiment, a state of the drug efficacy can be evaluated irrespective of a genotype of a patient. Therefore, it is unnecessary to detect the status of somatic mutation in the tumor tissue, and the method is clinically useful for predicting a therapeutic effect of a chemotherapeutic agent. As described above, evaluating whether chemotherapy is working appropriately by using the actual DNA amount in blood as an indicator instead of the status of cancer-related genes such as KRAS in the tumor tissue, is the finding firstly found by the inventors of the present invention. In particular, there is no specific nucleic acid marker usable for predicting the efficacy of a VEGF (vascular endothelial cell growth factor) inhibitor yet. However, it is also possible to predict a therapeutic effect of the VEGF inhibitor by using the method for evaluating a health condition according to the present embodiment.

In the quantification step of the method for evaluating a health condition according to the present embodiment, a method for measuring the amount of cfDNA per unit amount of a body fluid sample is not particularly limited. The method can be appropriately selected from methods used for quantitative detection of DNA and used. Examples of such methods include an absorbance method, an intercalation method, a real-time PCR method, a digital PCR method, a next generation sequencing method, an electrochemical detection method, and the like. These methods can be carried out in the usual manner.

The absorbance method, the intercalation method, and the electrochemical detection method are the most versatile and simple methods for DNA measurement. In a case where DNA concentration is measured by the absorbance measurement, it is preferable to use DNA purified from a body fluid sample. Extraction and purification of DNA from a body fluid sample can be carried out in the usual manner, and a commercially available common extraction kit and purification kit can be used. Examples of a fluorescent intercalator used in the intercalation method include PicoGreen, SYBR Green, ethidium bromide, thiazole orange, oxazole yellow, and the like.

In addition, the real-time PCR method and the digital PCR method are preferable in that quantitative detection of DNA can be performed with high sensitivity. Moreover, according to the same theory as the digital PCR, it is also possible to quantitatively determine the amount of cfDNA using the next generation sequencer. However, it has already been reported that DNA in serum or plasma has been fragmented, and it is preferable to design primers so that a length of a PCR product is about 100 to 200 bp, or 100 bp or less.

For example, the amount of cfDNA in a blood sample can be determined by detection using the digital PCR. Particularly, by using the technology Droplet Digital PCR (ddPCR) of Bio-Rad Laboratories, Inc. (Hindson, et. al., Analytical Chemistry, 2011, vol. 83 (22), pp. 8604-8610) and a digital PCR apparatus, “RainDrop Digital PCR System” of RainDance Technologies, Inc., the detection can be performed with high sensitivity. The more the number of droplets, the higher the accuracy of analysis. In order to ensure a sufficient level of detection sensitivity, it is preferable to define a surfactant concentration in a PCR master mix. For example, it is preferable that a final concentration of ethylene glycol or glycerol used as a preservative solution for DNA elongation enzyme and the like is 0.15% or less, and that a final concentration of Triton-X is 0.0003% or less. In a case where the surfactant concentration is equal to or more than the above-described final concentration, the number of emulsions drastically decreases and it becomes difficult to detect a PCR product with high sensitivity. In this case, a non-gene region or a gene region can be arbitrary selected for a gene region to be amplified.

Examples of another method include a method in which a primer in a specific region is set by the real-time PCR or the like using a nucleic acid in a blood sample as a template, and for example, a fragment containing a region that codes for a TCF4 gene is amplified, and then a probe capable of specifically hybridizing to a specific genotype of the TCF4 is brought into contact with this amplified product to detect whether or not an aggregate has been formed, with high sensitivity. It is possible to quantify the amount of cfDNA by performing the above method.

The probe is labeled so as to be detectable by, for example, a radioactive isotope (3H, 32P, 33P, and the like), a fluorescent agent (rhodamine, fluorescein, and the like), or a color former. In addition, the probe may be an antisense oligomer such as PNA, morpholino-phosphoramidates, and LNA. A base length of the probe is about 8 nucleotides to about 100 nucleotides, preferably about 10 nucleotides to about 75 nucleotides, more preferably about 15 nucleotides to about 50 nucleotides, and still more preferably about 20 nucleotides to about 30 nucleotides.

The method for evaluating a health condition according to the present embodiment can be more easily performed by making a reagent a kit form, the reagent being used for measuring the amount of cfDNA in a body fluid sample. The kit may contain protocols on the method for measuring the amount of cfDNA in a body fluid sample, a document in which a reference value and an evaluation method used for evaluating a health condition based on the cfDNA amount obtained are described, and the like.

A body fluid sample to be subjected to the quantification step of the method for evaluating a health condition according to the present embodiment is not particularly limited as long as the sample is a body fluid that can be expected to contain cfDNA. Examples thereof include blood, serum, plasma, urine, saliva, semen, thoracic exudate, cerebrospinal fluid, tears, sputum, mucus, lymph fluid, cytosol, ascites, pleural effusion, amniotic fluid, bladder lavage solution, bronchoalveolar lavage solution, and the like. As the body fluid sample to be used in the present embodiment, blood, serum, or plasma is preferable, and serum or plasma is more preferable. In the present embodiment, a sample obtained by a biopsy collection treatment such as resection of a primary tumor is not needed, and therefore collection of the sample can be performed less invasively.

In addition, in the method for evaluating a health condition according to the present embodiment, the cfDNA amount is used as an indicator. Therefore, it is possible to reduce the necessary amount of a body fluid sample to be smaller than that of a method in which a small amount of somatic mutation is detected. For example, in plasma or serum, the amount of the cfDNA can be measured if there is 1 mL of a sample. From this viewpoint, the method for evaluating a health condition according to the present embodiment is clinically favorable because of the small burden on a subject.

The body fluid sample to be used in the present embodiment may be any sample collected from a subject after tumor resection, and the types of a tumor of the subject is not particularly limited. In addition, the tumor may be a primary tumor, a metastatic tumor, or a recurrent tumor. Furthermore, the tumor may exist in a plurality of places in the body of the subject. Such tumors include cancer of brain, liver, kidney, bladder, breast, stomach, ovary, colorectum, prostate, pancreas, lung, vulva, thyroid, and esophagus, sarcoma, gliosarcoma, head and neck cancer, leukemia, and lymphoid malignancies. More specific examples thereof include neuroblastoma, intestinal cancer (for example, rectal cancer, colorectal cancer, familial polyposis coli cancer, and hereditary nonpolyposis colorectal cancer), esophageal cancer, lip cancer, laryngeal cancer, hypopharyngeal cancer, tongue cancer, salivary gland cancer, gastric cancer, adenocarcinoma, medullary thyroid cancer, thyroid artery papillary cancer, kidney cancer, renal parenchymal cancer, ovarian cancer, cervical cancer, uterine cancer, endometrial cancer, choriocarcinoma, pancreatic cancer, prostate cancer, testicular cancer, breast cancer, ureter cancer, melanoma, brain cancer (for example, glioblastoma, astrocytoma, meningioma, medulloblastoma, and peripheral neuroectodermal tumor), Hodgkin's lymphoma, non-Hodgkin's lymphoma, Burkitt's lymphoma, acute lymphocytic leukemia (ALL), chronic lymphocytic leukemia (CLL), acute myeloid leukemia (AML), chronic myeloid leukemia (CML), adult T-cell leukemia, hepatocellular carcinoma, gallbladder cancer, bronchial cancer, small cell lung cancer, non-small cell lung cancer, multiple myeloma, basal cell carcinoma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing's sarcoma, and plasmacytoma. As a resected tumor of a subject from which a body fluid sample is collected, which is provided for the present embodiment, metastatic medulloblastoma, gastrointestinal stromal tumor, dermatofibrosarcoma protuberans, colon rectal cancer, colorectal cancer, lung cancer, non-small-cell lung cancer, small cell lung cancer, chronic myeloproliferative disease, acute myelogenous leukemia, thyroid cancer, pancreatic cancer, bladder cancer, kidney cancer, melanoma, breast cancer, prostate cancer, ovarian cancer, cervical cancer, head and neck cancer, brain tumor, hepatocellular carcinoma, hematologic malignancy, or pre-cancer causing these cancers is preferable, and colorectal cancer, colon rectal cancer, lung cancer, hepatocellular carcinoma, breast cancer, ovarian cancer, prostate cancer, kidney cancer, head and neck cancer, or cervical cancer is more preferable.

In the evaluation step, the test cfDNA amount obtained in the quantification step is compared with a reference value to evaluate a health condition of the subject. Specifically, in a case where the test amount of cfDNA per unit amount of the body fluid sample of the subject is higher than the reference value, it is evaluated that in the subject, a tumor has increased or new metastasis has occurred or there is a high possibility of an increase of a tumor or occurrence of new metastasis, or that in the subject, while a tumor has not increased and no new metastasis has occurred, the health condition has deteriorated or there is a high possibility of deterioration of the health condition.

The reference value is any one of the following (i) to (iii).

(i) A predetermined threshold value

(ii) An amount of cfDNA per unit amount of a body fluid sample collected before tumor resection from a subject, or an amount of cfDNA per unit amount of a body fluid sample collected from a subject within a period from 1 week to 3 months after tumor resection

(iii) An amount of cfDNA per unit amount of a body fluid sample collected from a subject by the time of collecting a body fluid sample

The threshold value of (i) can be experimentally set. For example, a body fluid sample is collected from a group of cancer patients whose tumor recurrence or metastasis is confirmed to occur by another diagnostic method. In addition, a body fluid sample is collected from a group of cancer patients whose tumor recurrence or metastasis is confirmed not to occur by another diagnostic method, or a group of healthy subjects. By measuring the amount of cfDNA per unit amount in the body fluid samples of both groups under the same measurement condition and comparing the measurement values of both groups, the threshold value for discriminating between the two groups can be appropriately set. A group of patients suffering from diseases other than cancer may be used instead of the group of healthy subjects. In addition, a group of cancer patients before tumor resection treatment in which onset of cancer is confirmed may be used instead of the group of cancer patients whose tumor recurrence or metastasis is confirmed to occur by another diagnostic method.

For example, in a case where the body fluid sample is plasma and in a case where 500 ng of cfDNA per mL of plasma is detected, there is a possibility that tumor recurrence or metastasis occurs. Furthermore, in a case where a cfDNA value is 750 ng, the possibility thereof becomes higher, and in a case where a cfDNA value exceeds 1000 ng, the certainty thereof increases. Therefore, 1000 ng of a cfDNA value per mL of plasma can be used as a reference value. In this case, in a case where a cfDNA value per mL of plasma of a subject after tumor resection exceeds 1000 ng, it is evaluated that in the subject, tumor recurrence or metastasis occurs or a possibility thereof is high. The reference values for plasma and serum are almost equal.

In addition, the abundance of tumor tissue in the body is decreased by the tumor resection treatment, but the tumor resection treatment is highly invasive and is a heavy burden on the body of the subject. Therefore, in general, the amount of cfDNA in a body fluid increases in a short term immediately after the tumor resection treatment and then stabilizes at a low value when the influence of the resection treatment becomes weaker. As the reference value used in the evaluation step, the amount of cfDNA per unit amount of the body fluid sample when the body is recovered from the influence of the resection treatment, and recurrence, metastasis, or the like of the tumor has not yet occurred, can be used. Recovery from the influence of the resection treatment requires at least 1 week in many cases, and the body usually recovers from the influence of the resection treatment between 1 month and 3 months after the tumor resection. The amount of cfDNA per unit amount of the body fluid sample collected within this period can be used as a reference value used in the evaluation step. As the reference value of (ii) in the present embodiment, the amount of cfDNA per unit amount of the body fluid sample collected from the subject between 1 week and 3 months after the tumor resection can be used. The amount of cfDNA per unit amount of the body fluid sample is collected from the subject preferably between 1 month and 3 months after the tumor resection, more preferably 40 days to 3 months after the tumor resection, and still more preferably 42 to 90 days after the tumor resection. However, depending on the subjects, in some cases, the amount of cfDNA in the body fluid before the tumor resection treatment may be lower than the amount of cfDNA in the body fluid after the tumor resection treatment and after the body has recovered. In this case, it is preferable to use the amount of cfDNA per unit amount of the body fluid sample collected from the subject before the tumor resection as a reference value to be used in the evaluation step ((ii) above).

With respect to the amount of cfDNA in the subject when about 1 month or more have passed since the tumor resection treatment, an increase in the amount of cfDNA due to the influence of surgical invasion is ended. Therefore, in subjects whose cfDNA amount in the body fluid has not decreased during this period, it is presumed that some sort of disease action has occurred and thus a deterioration of the health condition has occurred. In other words, in the method for evaluating a health condition according to the present embodiment, it is also possible to use the body fluid sample collected from the subject within 1 to 3 months from the tumor resection treatment, and the value of (i) for example as a reference value. In this case, in a case where the quantitatively determined amount of cfDNA per unit amount of the body fluid sample is higher than the reference value, it can be evaluated that in the subject, some sort of disease action has occurred and thus a deterioration of the health condition has occurred or there is a high possibility that a deterioration thereof occurs. Based on evaluation, it is possible to provide therapy options of doctors which include whether additional imaging of CT is required, and whether there are opinions on side effects, and the like.

As the reference value used in the evaluation step, it is also possible to use the amount of cfDNA per unit amount of the body fluid sample previously collected from the same subject ((iii) above). As the reference value, a sample collected after the tumor resection is preferable. For example, by using the amount of cfDNA per unit amount of the body fluid sample collected 3 months after the tumor resection treatment as a reference, it is possible to evaluate body fluid sample corrected after the collection of the body fluid sample of 3 months after the tumor resection treatment.

It is preferable to detect the presence, absence, and the like of recurrence or metastasis of a cancer patient as soon as possible after tumor resection. It is preferable that the method for evaluating a health condition according to the present embodiment is performed with regard to the body fluid sample collected from the subject over time, the amount of cfDNA per unit amount of the body fluid sample of the subject is monitored, and a health condition of the subject is evaluated over time. In a case where the amount of cfDNA per unit amount of the body fluid sample of the subject is increasing, it can be evaluated that in the subject, a tumor has increased or new metastasis has occurred or there is a high possibility of an increase of a tumor or occurrence of new metastasis, or while a tumor has not increased and no new metastasis has occurred, the health condition has deteriorated or there is a high possibility of deterioration of the health condition. In cancer therapy, blood sampling for a tumor marker test is usually performed about once a month. Therefore, it is also possible to perform the method for evaluating a health condition according to the present embodiment over time by using the remained blood sample for the tumor marker test. It is sufficient for the present embodiment to quantitatively determine the amount of cfDNA in serum or plasma unlike a so-called genetic test, and therefore it is easy to clinically practice the present embodiment in the viewpoint that only a small amount of a sample is needed.

In a case where the subject is receiving chemotherapy, tumor growth or new metastasis means that the chemotherapy is not effective. Therefore, in a case where the subject is receiving chemotherapy after tumor resection and in a case where the test amount of cfDNA measured in the quantification step is higher than the reference values of any one of (i) to (iii), it can be evaluated that the chemotherapy is not exhibiting the effect.

Even in a case where tumor growth or new metastasis does not occur, the amount of cfDNA in a body fluid increases if the health condition deteriorates due to side effects caused by therapeutic practice and the like. In a case where the subject is receiving chemotherapy after tumor resection and in a case where the test amount of cfDNA measured in the quantification step is higher than the reference values of any one of (i) to (iii), it can be evaluated that side effects due to the chemotherapy has occurred or there is a high possibility of occurrence of side effects due to the chemotherapy.

In anticancer therapy, severe side effects in chemotherapy are particularly problematic in many cases. In particular, severe skin disorder and depilation. (interstitial) pneumonia, peritonitis, or the like are caused as side effects. As a result, there are cases where it is difficult to continue drug therapy thereafter, and it is necessary to determine drug withdrawal in many cases. Prediction and evaluation of side effects are also important for doctors to measure the timing of the drug withdrawal, but clinically applicable biomarkers for prediction of side effects or for early detection has not yet been known in the related art. In the method for evaluating a health condition according to the present embodiment, prediction of the risk of side effects causing deterioration of a health condition and early detection are possible by using the cfDNA amount as an indicator. For example, in a case where the body fluid sample is plasma and the amount of 1000 ng of a cfDNA per mL of plasma is used as a reference value, and in a case where the amount of a cfDNA per mL of plasma of a subject receiving chemotherapy after tumor resection exceeds 1000 ng, it can be evaluated that in the subject, side effects caused by the chemotherapy occurs or there is a high possibility that side effects will occur. The evaluation obtained is useful as information (material) for determining whether to continue or stop chemotherapy, or to change a chemotherapeutic agent to be used. For example, in a case where there is a tendency that the amount of cfDNA per mL of plasma increases up to near 1000 ng or exceeds 1000 ng, subsequent therapy is stabilized by drug withdrawal, and thus anticancer therapy becomes easy to be performed. As described above, the evaluation obtained by the present embodiment is clinically extremely useful as a predictive indicator that can suggest drug withdrawal. In a case where the subject has been administered a chemotherapeutic agent in the past, it is preferable to use, as the reference value used in the evaluation step, the amount of cfDNA per unit amount of a body fluid sample collected 60 days after the administration of the chemotherapeutic agent.

The chemotherapeutic agent which is used in the chemotherapy received by the subject and on which side effects and an efficacy state are evaluated in the method for evaluating a health condition according to the present embodiment is not limited and may have a compound having cytotoxicity or cell division inhibition. Specific examples thereof include (i) an antimetabolite such as fluorouracil, capecitabine, cytarabine, fludarabine, 5-fluoro-2′-deoxyuridine, tegafur/gimeracilioteracil potassium (TS-1), gemcitabine, hydroxyurea, or methotrexate; (ii) a DNA fragmenting agent such as bleomycin: (iii) a DNA crosslinking agent such as chlorambucil, cisplatin, cyclophosphamide, or nitrogen mustard; (iv) an intercalating agent such as adriamycin (doxorubicin) or mitoxantrone; (v) a protein synthesis inhibitor such as L-asparaginase, cycloheximide, puromycin, or diphtheria toxin; (vi) a topoisomerase I poison such as camptothecin or topotecan; (vii) a topoisomerase II poison such as etoposide (VP-16) or teniposide; (viii) a microtubule-associated agent such as colcemid, colchicine, paclitaxel, vinblastine, or vincristine; (ix) a kinase inhibitor such as flavopiridol, staurosporine, STI 571 (CGP 57148B), or UCN-01 (7-hydroxystaurosporine); (x) various investigational drugs such as thioplatin, PS-341, phenylbutyrate. ET-18-OCH3, or farnesyl transferase inhibitors (L-739749, L-744832): polyphenols such as quercetin, resveratrol, piceatannol, epigallocatechin gallate, theaflavins, flavanols, procyanidins, and betulinic acid and a derivative thereof; (xi) a hormone such as glucocorticoid or fenretinide; and (xii) an antihormone such as tamoxifen, finasteride, or LHRH antagonist. Examples of the chemotherapeutic agent further include folinic acid (leucovorin), oxaliplatin, irinotecan, daunarubicin, taxotere, and mitomycin C. Examples of the chemotherapeutic agent still further include a molecular-targeted agent such as cetuximab, panitumumab, bevacizumab, gefitinib, erlotinib, regorafenib, crizotinib, sunitinib, sorafenib, everolimus, trastuzumab, lapatinib, and rituximab. These chemotherapeutic agents may be used alone, or two or more kinds thereof may be used in combination. The chemotherapeutic agents on which side effects and an efficacy state are evaluated in the present embodiment are preferably one or more selected from fluorouracil, leucovorin, oxaliplatin, capecitabine, tegafur/gimeracilloteracil potassium, irinotecan, bevacizumab, cetuximab, and panitumumab.

The subject may receive another antitumor therapy other than chemotherapy. Specific examples of the other antitumor therapy include radiation therapy and the like. In addition, the subject may have received, in the past, therapy using a chemotherapeutic agent different from the chemotherapeutic agent to be evaluated.

For example, in a case where the amount of cfDNA per unit amount of plasma collected from a subject when 40 to 90 days have passed after the tumor resection is used as a reference value and in a case where the amount of cfDNA per unit amount of plasma collected from the subject is higher than the reference value, it can be evaluated that there is a high possibility that the current therapy is not effective. That is, it can be evaluated that there is a high possibility that recurrence of cancer, that is, metastasis or recurrence have occurred, or side effects that cannot be overlooked by the current therapy occur. In a case where such an evaluation is obtained, it is preferable to additionally perform a CT imaging test or the like to the subject. On the other hand, in a case where the amount of cfDNA in the body fluid sample is sequentially monitored, and in a case where the amount of cfDNA is stably low, specifically, the amount thereof is 1000 ng or less per mL of plasma, or in a case where the amount of cfDNA is kept in a lower state than that before surgery, it can be evaluated that a chemotherapeutic agent used in the current therapy is exhibiting the effect or there is a high possibility that the risk of metastasis or recurrence is small and there is not any side effect state caused by the chemotherapeutic agent.

Method for Predicting Long-Term Efficacy of Anticancer Agent

As described above, the amount of cfDNA per unit amount in a body fluid is obtained as an indicator for evaluating or predicting the efficacy of therapy of chemotherapy such as an anticancer agent. In other words, long-term efficacy of an anticancer agent can be predicted by using, as an indicator, the amount of cfDNA per unit amount in a body fluid of a subject who received anticancer agent therapy. The term “long-term efficacy of an anticancer agent” means that in a subject administered with an anticancer agent, an increase in volume, metastasis, or recurrence of tumor tissue does not occur for at least 6 months.

That is, the method for predicting the long-term efficacy of an anticancer agent according to the present embodiment (hereinafter may be referred to as “prediction method according to the present embodiment”) includes a monitoring step of measuring the amount of cfDNA per unit amount of a body fluid sample collected from a subject through two or more collections over time so as to monitor the amount of cfDNA per unit amount of the body fluid sample of the subject; and a prediction step of comparing the amount of cfDNA obtained in the monitoring step with a predetermined reference value so as to predict whether or not long-term efficacy of an anticancer agent with respect to the subject is obtained.

In a state where the anticancer agent exhibits long-term efficacy and a worsening of a tumor is suppressed, the amount of cfDNA per unit amount of a body fluid sample of a cancer patient tends to be small. In addition, in a state where the anticancer agent is not exhibiting efficacy and the tumor becomes worse, the cfDNA amount tends to increase. In the prediction step, in a case where the amount of cfDNA per unit amount of the body fluid sample of the subject decreased from a state exceeding the reference value to the reference value or less, or in a case where the cfDNA amount of all body fluid samples is equal to or less than the reference value, it is predicted that long-term efficacy of the anticancer agent that was taken by the subject will be obtained. On the contrary, in a case where the amount of cfDNA per unit amount of the body fluid sample of the subject increases from a state equal to or lower than the reference value to a state exceeding the reference value, or in a case where the cfDNA amount of all body fluid samples exceeds the reference value, it is predicted that long-term efficacy of the anticancer agent administered to the subject cannot be obtained.

For example, the reference value used in the prediction step can be set experimentally in advance.

For example, among cancer patients taking the anticancer agent, a body fluid sample is collected from a group of cancer patients, in which tumor tissue growth, recurrence, or metastasis does not occur and the anticancer agent is determined to be effective by other diagnostic methods. In addition, a body fluid sample is collected from a group of cancer patients, in which any one of tumor tissue growth, recurrence, or metastasis occurs and the anticancer agent is not determined to be effective by other diagnostic methods. By measuring the amount of cfDNA per unit amount in the body fluid samples of the both groups under the same measurement condition, and comparing the measured values of the both groups, it is possible to appropriately set a threshold value for discriminating between the two groups.

For example, in a case where the body fluid sample is serum, the amount of 20 ng of a cfDNA per mL of serum can be used as a reference value. In this case, in a case where a cfDNA value per mL of serum of a subject exceeds 20 ng, it is predicted that long-term efficacy cannot be obtained with respect to the anticancer agent taken by the subject.

The prediction in the prediction step can be used as a material for determining whether the subject is to continue or stop the anticancer agent, or to change the anticancer agent to be used. For example, when determining whether to continue using the anticancer agent to a subject who is receiving anticancer agent therapy, in a case where the anticancer agent is predicted to exhibit long-term efficacy in the prediction step, the anticancer agent can be determined to be continuously used. In addition, in a case where it is predicted that the anticancer agent does not exhibit long-term efficacy, it can be determined that the use of the anticancer agent is stopped or the anticancer agent is changed to another anticancer agent.

In the prediction method according to the present embodiment, the subject to be predicted for long-term efficacy of the anticancer agent may be a patient to whom an anticancer agent including a molecular therapeutic agent is administered. The types of the affected tumor are not particularly limited, and the tumor may be a tumor existing in a plurality of places in the body. In addition, the tumor may be a primary tumor, a metastatic tumor, or a recurrent tumor. Examples of the types of the tumor include exemplary examples such as a tumor from which a subject of the method for evaluating a health condition according to the present embodiment suffers. As the subject to be predicted in the prediction method according to the present invention, a subject who suffers from one or two or more selected from the group consisting of colorectal cancer, colon cancer, rectal cancer, lung cancer, liver cancer, breast cancer, ovarian cancer, prostate cancer, renal cancer, esophageal cancer, head and neck cancer, uterine cancer, and cervical cancer, is preferable.

In the prediction method according to the present embodiment, the anticancer agent to be predicted for long-term efficacy is not particularly limited. Examples of the anticancer agent include the agent same as the chemotherapeutic agent used in chemotherapy received by the subject and on which side effects and an efficacy state are evaluated in the method for evaluating a health condition according to the present embodiment.

Among these, the prediction method according to the present embodiment is preferably used for predicting the long-term efficacy of one or more anticancer agents selected from the group consisting of the EGFR inhibitor and the VEGF inhibitor.

In the prediction method according to the present embodiment, the body fluid sample to be provided may be the blood itself collected from the subject. Serum or plasma is particularly preferable. As a blood sample, it is possible to use the remained blood sample collected for a tumor marker test in cancer therapy. However, during the monitoring step, it is preferable not to change the kind of body fluid sample to be provided.

Examples of the method for measuring the amount of cfDNA per unit amount of a body fluid sample in the monitoring step of the prediction method according to the present embodiment, include those exemplified as a method for measuring the amount of cfDNA in the method for evaluating a health condition according to the present embodiment.

### EXAMPLES

Hereinafter, the present invention will be specifically described with reference to examples, but the present invention is not limited to the following examples. The following tests were approved by the Ethics Review Committee of Nippon Medical School Hospital and informed consent including this study was obtained from all patients.

### Example 1

With respect to 6 patients having colorectal cancer (A to F) from whom a primary tumor had already been resected and a new metastatic tumor (liver) was surgically resected, a concentration of cfDNA contained in plasma prepared from blood collected over time before and after the surgery was fluorescent measured by using a commercially available kit (product name: Qubit 2.0 (manufactured by Life Technologies)), and therefore the concentration of cfDNA was calculated. Based on the obtained value, an amount of cfDNA per mL of plasma was calculated. The details will be described below.

Clinical Sample

6 to 9 mL of peripheral blood of patients with recurrent colorectal cancer, from whom a primary tumor had already been resected and a new metastatic tumor (liver) was surgically resected, was collected before and after the surgery. The obtained blood was centrifuged (1500 rpm, 10 minutes). The resulting crude plasma component was further centrifuged (1500 rpm, 10 minutes). Thereafter, the supernatant portion from which the cell fragments had been removed was recovered and used as a plasma sample.

Measurement of CEA and CA-19-9 in Plasma

CA-19-9, and CEA in plasma was measured by a CLEIA method (chemiluminescence enzyme immunoassay method). A reference value of CA-19-9 was set to be 37 U/mL or less, and a reference value of CEA was set to be 5.0 ng/mL or less.

Isolation and Purification of cfDNA from Plasma

Isolation and purification of cfDNA from plasma were carried out using QIAamp Circulating Nucleic Acid Kit (manufactured by Qiagen). The amount of a plasma sample provided in this kit was 1 mL. The isolation and purification steps of DNA were performed in accordance with the instructions attached to the kit. Final elution from a spin column was performed using 50 μL of a TE buffer.

Quantitative Determination of cfDNA

Quantitative determination of cfDNA was performed using Qubit (registered trademark) Fluorometer (manufactured by Life Technologies). For all samples to be measured, the isolated DNA was diluted 200 times with a predetermined reaction solution and used.

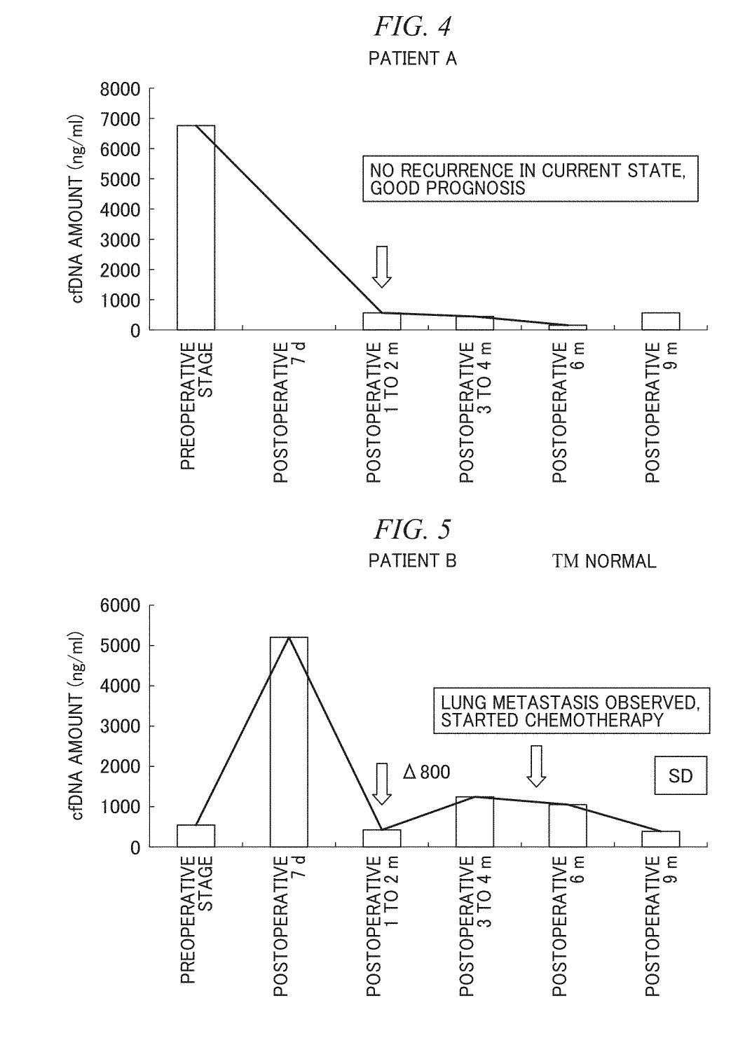

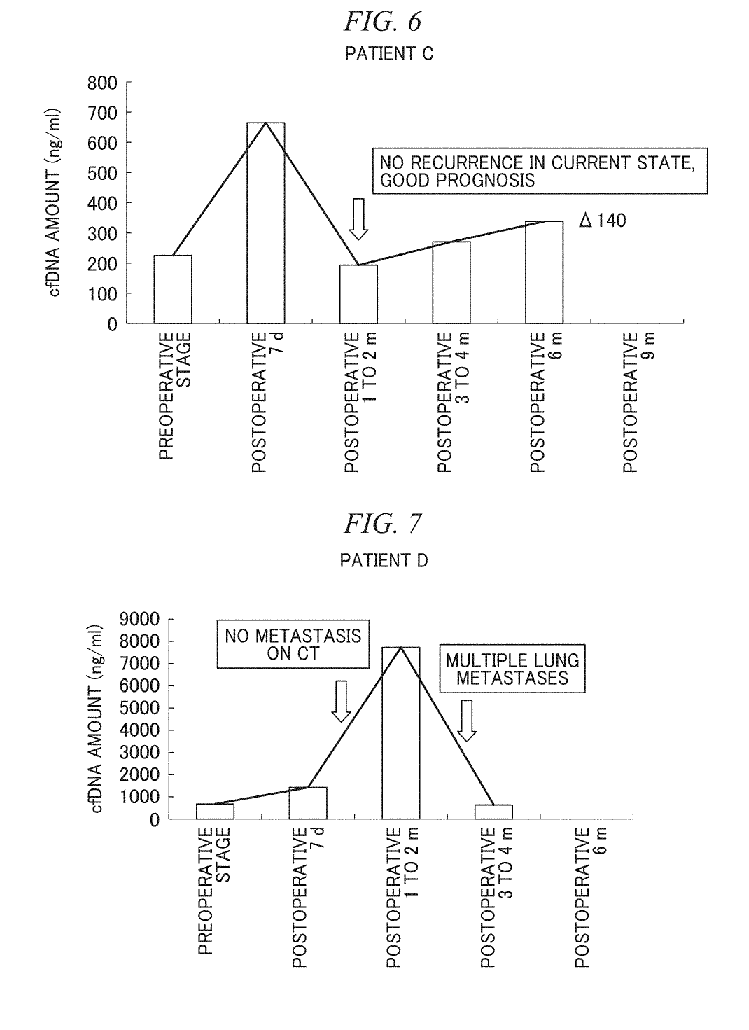

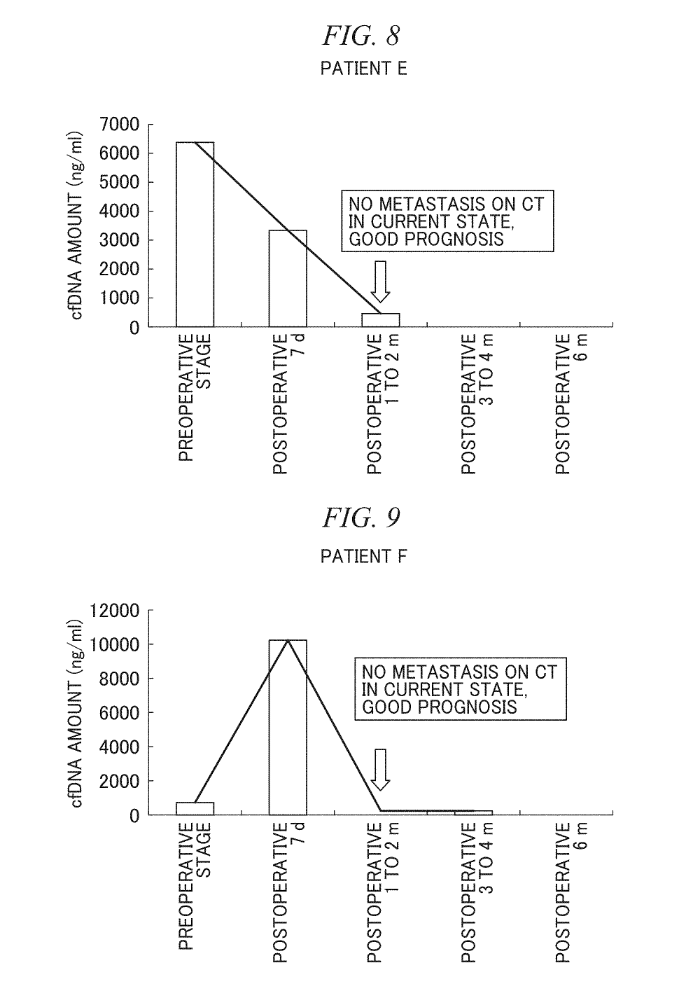

Patient information and measurement results of the cfDNA amount and the like in this experiment are shown in Table 1. In addition, FIGS. 4 to 9 show the amount of DNA of each patient over time. In Table 1, “postoperative 7 d” shows the result of blood sampling near the 7th day after the liver resection. In Table 1, “postoperative 1 to 2 m”, “postoperative 3 to 4 m”, “postoperative 6 m”, and “postoperative 9 m” show the result of blood sampling near 1 to 2 months elapsed after the liver resection, near 3 to 4 months elapsed after the liver resection, near 6 months elapsed after the liver resection, and near 9 months elapsed after the liver resection, respectively.

TABLE 1

| Patient ID | | A | B | C | D | E | F |
|---|---|---|---|---|---|---|---|
| Preoperative | Date | 2014 May 14 | 2014 Jun. 25 | 2014 Aug. 7 | 2014 Aug. 27 | 2014 Oct. 17 | 2014 Oct. 8 |
| stage | | | | | | | |
| | DNA (ng/mL) | 6720 | 488 | 334 | 690 | 6400 | 786 |
| | CEA (ng/mL) | 3.0 | 13.9 | 11.7 | 36.5 | 4.9 | 3.3 |
| | CA19-9 (U/mL) | 7.9 | 4.7 | 32.0 | 120000 | 5.3 | 19.3 |
| Postoperative | Date | — | 2014 Jul. 2 | 2014 Aug. 13 | 2014 Sep. 3 | 2014 Oct. 27 | 2014 Dec. 8 |
| 7 d | | | | | | | |
| | DNA (ng/mL) | — | 5240 | 682 | 1400 | 3380 | 10300 |
| | CEA (ng/mL) | — | — | — | 16.7 | 4.0 | 3.9 |
| | CA19-9 (U/mL) | — | — | — | 12000 | 6.4 | 20.2 |
| Postoperative | Date | 2014 Aug. 20 | 2015 Aug. 26 | 2014 Oct. 8 | 2014 Dec. 8 | 2015 Jan. 15 | 2015 Jan. 8 |
| 1 to 2 m | | | | | | | |
| | DNA (ng/mL) | 546 | 438 | 2120 | 7760 | 580 | too low |
| | CEA (ng/mL) | 2.8 | 3.7 | 2.8 | 88.2 | 5.0 | 21.0 |
| | CA19-9 (U/mL) | 3.8 | 7.2 | 7.8 | 12000 | 3.2 | 22.5 |
| Postoperative | Date | 2014 Nov. 5 | 2014 Nov. 11 | 2014 Dec. 3 | 2015 Mar. 11 | 2015 Apr. 9 | 2015 Feb. 26 |
| 3 to 4 m | | | | | | | |
| | DNA (ng/mL) | 502 | 1220 | 340 | 524 | — | 240 |
| | CEA (ng/mL) | 3.5 | 3.8 | 3.3 | 42.7 | — | 9.3 |
| | CA19-9 (U/mL) | 4.6 | 3.7 | 7.7 | 8443 | — | 27.6 |
| Postoperative | Date | 2015 Jan. 7 | 2015 Jan. 13 | 2015 Feb. 4 | — | — | — |
| 6 m | | | | | | | |
| | DNA (ng/mL) | 176 | 1060 | 1150 | — | — | — |
| | CEA (ng/mL) | 3.1 | 5.0 | 3.7 | — | — | — |
| | CA19-9 (U/mL) | 5.2 | 3.3 | 7.0 | — | — | — |
| Postoperative | Date | 2015 Feb. 4 | 2015 Mar. 3 | — | — | — | — |
| 9 m | | | | | | | |
| | DNA (ng/mL) | 524 | 408 | — | — | — | — |
| | CEA (ng/mL) | 2.5 | 3.5 | — | — | — | — |
| | CA19-9 (U/mL) | 3.4 | 4.6 | — | — | — | — |

| | | | | | | |
|---|---|---|---|---|---|---|
| Date of liver resection | 2014 May 14 | 2014 Jun. 25 | 2015 May 14 | 2015 May 14 | 2015 May 14 | 2015 May 14 |
| KRAS | wild | wild | wild | unclear | G12D | wild |
| Outcome after liver resection | 2015 Feb. 4 | 2014 Sep. 30 | 2014 Dec. 3 | 2014 Nov. 28 | 2015 Jan. 15 | Postoperative |
| | No | Lung | No | No recurrence | No | CEA |
| | recurrence | metastasis; | recurrence | on CT; | recurrence | concentration |
| | on CT | 2014 November | on CT; | 2015 Jan. 7 | on CT | increased but |
| | | Started | 2015 Feb. 4 | PE repetition | | no recurrence |
| | | chemotherapy | No | and LN | | on CT on |
| | | | recurrence | recurrence | | 2015 Feb. 26 |
| | | | on CT | 2015 Jan. 28 | | |
| | | | | Started | | |
| | | | | chemotherapy | | |

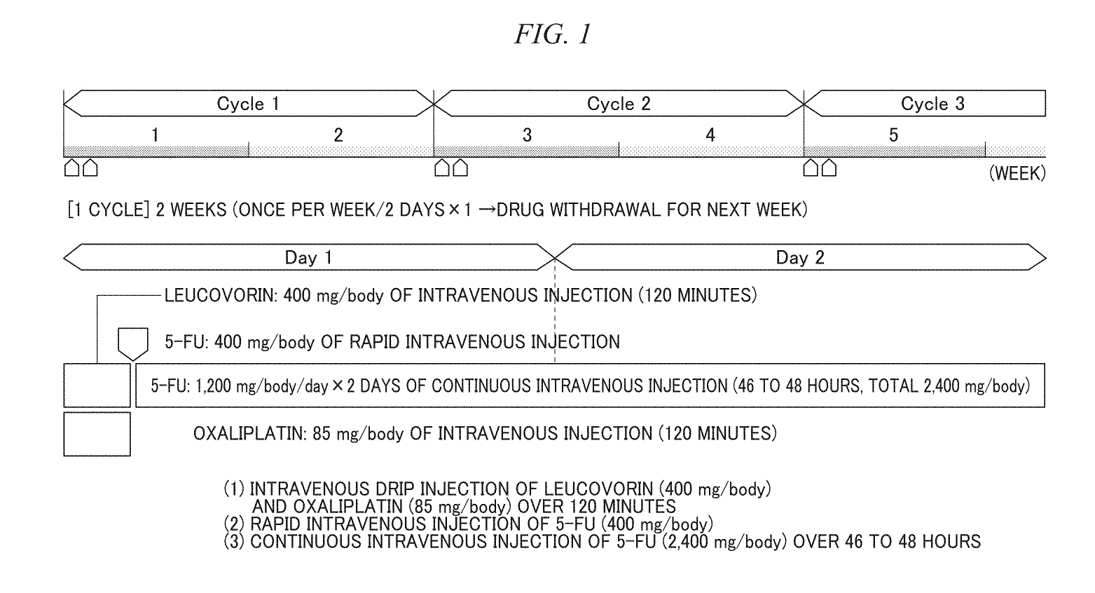

FOLFOX chemotherapy (fluorouracil/folinic acid/oxaliplatin) was performed for Patients A and C, both before the liver resection. Specifically, 400 mg/body of folinic acid (leucovorin) and 85 mg/body of oxaliplatin were administered by an intravenous drip injection over 2 hours. Thereafter, 400 mg/body of fluorouracil (5-FU) were administered by a rapid intravenous drip injection, and furthermore, 2,400 mg/body were administered by a continuous intravenous drip injection for 46 to 48 hours. The dosage regimen is shown in FIG. 1.

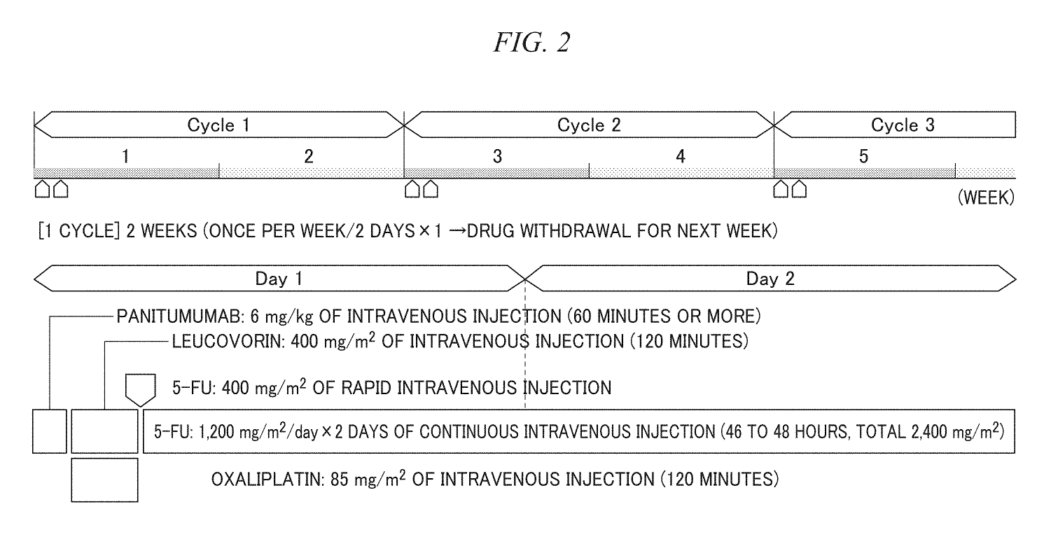

A combination therapy of mFOLFOX+panitumumab was performed for Patients B and F, both before the liver resection, with a regimen shown in FIG. 2. An intravenous drip injection of panitumumab (6 mg/kg) was performed over 60 minutes or more. Thereafter, intravenous drip injections of leucovorin (400 mg/m²) and oxaliplatin (85 mg/m²) were performed over 120 minutes. Subsequently, 5-FU (400 mg/m²) was rapidly injected intravenously, and 5-FU (2,400 mg/m²) was continuously injected intravenously over 46 to 48 hours. The combination therapy of mFOLFOX+panitumumab was performed for Patient B also after the liver resection with the same regimen as in FIG. 2 (“Started chemotherapy” in the table).

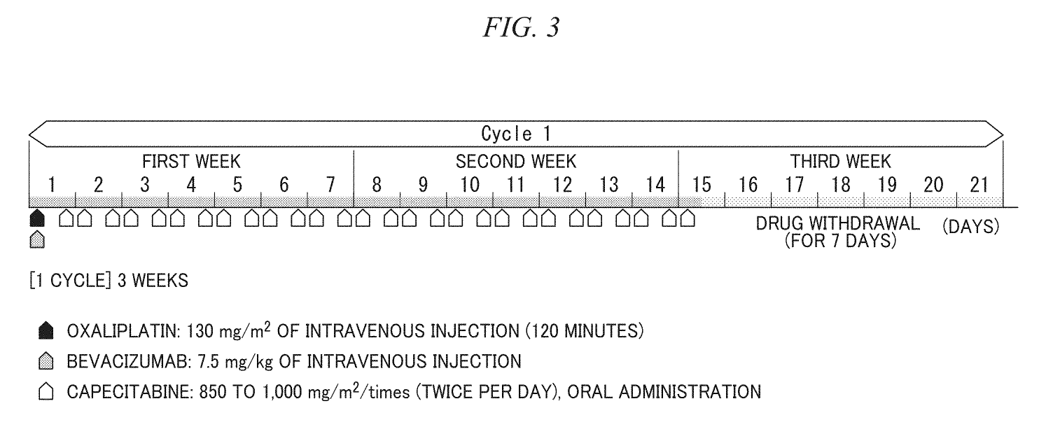

XELOX chemotherapy in combination with bevacizumab (oxaliplatin/capecitabine) was performed for Patient D (“Started chemotherapy (XELOX)” in the table). A regimen is shown in FIG. 3. On the first day, bevacizumab (7.5 mg/kg) was intravenously injected in the morning, followed by an intravenous injection of oxaliplatin (130 mg/m²) over 120 minutes or more. Furthermore, capecitabine (850 to 1000 mg/m²) was orally administered after dinner. The capecitabine (850 to 1000 mg/m²/times) was orally administered twice a day (morning and after dinner) from the 2nd to 14th days. From day 15 onwards, the capecitabine (850 to 1000 mg/m²) was orally administered only after breakfast. From 16th to 21st days was for a drug withdrawal, which was set as one cycle.

No chemotherapy was performed for Patient E.

Patients A, C, E, F (FIGS. 4, 6, 8, and 9) had good prognosis and anticancer agent therapy was also effective. Each amount of cfDNA near 1 to 2 months elapsed after the surgery was low, and no rapid increase was observed even in the follow-up thereafter.

The fact common to all patients was that a cfDNA concentration was high due to surgical invasion in about 1 week after the surgery, and at least 1000 ng of cfDNA was present in 1 mL of plasma Therefore, there is a high possibility of misjudgment if the amount of cfDNA at this time is used to compare the amount at this time and before or after thereof. Accordingly, it was confirmed that that the reference value used for the evaluation is preferably a measurement value of a body fluid sample collected when at least 1 week or more, preferably 30 days or more, and more preferably 40 days or more have been elapsed after the surgery.

On the other hand, postoperative recurrence occurred in Patient B (FIG. 5). The liver recurrence in Patient B was not confirmed by CT after the surgery. However, lung metastasis was observed near 1 to 2 months after the surgery, and the amount of cfDNA per mL of plasma increased by about 800 ng near 3 to 4 months after the surgery and the amount thereof exceeded 1000 ng per mL of plasma. That is, it was confirmed that the amount of cfDNA in plasma tends to increase in accordance with lung metastasis. Furthermore, the amount was observed to tend to decrease after the start of chemotherapy, which indicates that the amount of cfDNA in plasma is also responsive to the efficacy of chemotherapy. On the other hand, CEA and CA-19 consistently maintained normal values and did not respond to the metastasis at all. Based on these results, it became clear that even for the metastasis which cannot be detected by a test by a tumor marker of the related art such as CEA and CA-19, detection can be performed with high sensitivity by using the amount of cfDNA in a body fluid as an indicator.

In Patient D (FIG. 7), metastasis was not confirmed on CT performed in November 2014. However, in the subsequent PET-CT performed on January 2015, metastasis was confirmed. In Patient D, the amount of cfDNA per mL of plasma increased from near 1 to 2 months after the surgery, which is before the metastasis is confirmed, and the amount was observed to tend to decrease after the start of chemotherapy. In other words, a significant increase in cfDNA amount was observed one month before CT imaging. Although CEA and CA-19 showed abnormal values before the surgery, the values hardly changed after the surgery and remained constant, and there was no response to the metastasis at all. Also based on this result, it became clear that an increase in the amount of cfDNA in a body fluid can predict tumor metastasis or recurrence.

As above, there is a possibility that the cfDNA amount in a cancer patient reflects a real-time tumor state, and it became clear that a steady response to metastasis can be secured rather than at least tumor markers such as CEA and CA-19.

### Example 2

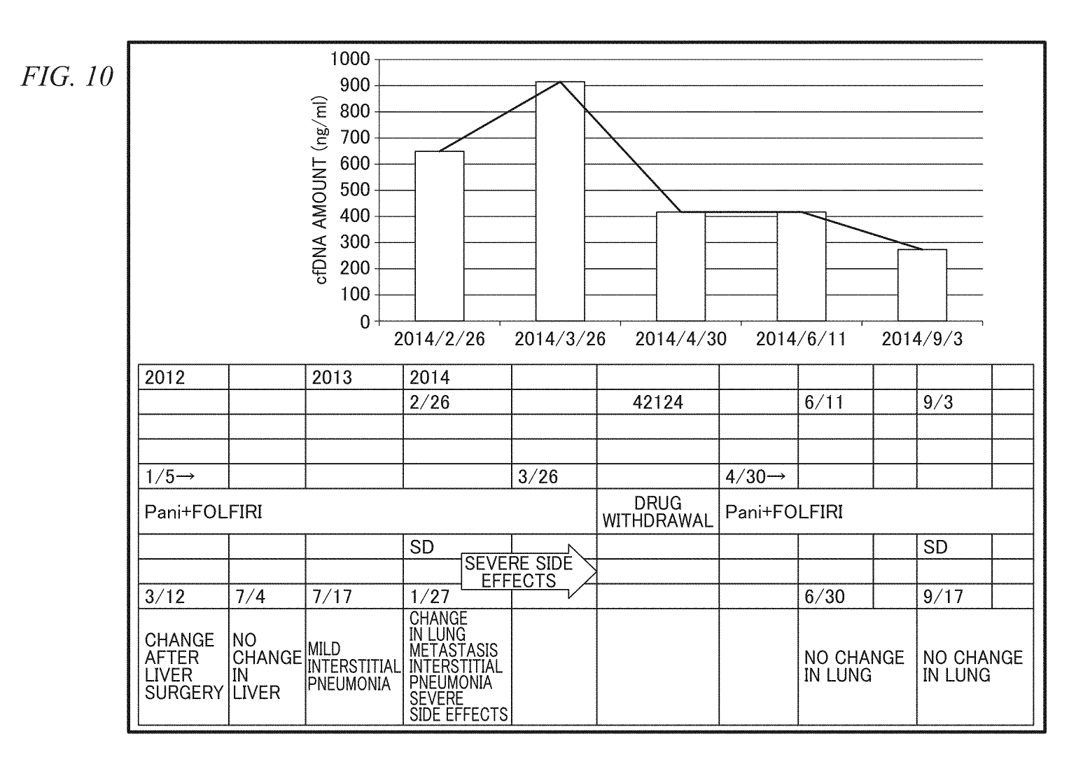

With respect to a patient (Patient G) who is different from the patient of Example 1 and has colorectal cancer from which a primary tumor (liver) was surgically resected, blood was sampled over time and the amount of cfDNA per mL of plasma was measured. The cfDNA amount was measured in the same manner as in Example 1. Clinical information on Patient G and changes in cfDNA amount (ng) per mL of plasma are shown in FIG. 10. As in Patient B of Example 1, a combination therapy of mFOLFOX+panitumumab was performed for the patient with the regimen shown in FIG. 2, but pneumonia was caused by side effects. In accordance with the severity of pneumonia, the amount of cfDNA increased in response, but when the drug was withdrawn, pneumonia in the patient was improved and the amount of cfDNA also decreased at a stroke. After pneumonia was improved, the combination therapy of mFOLFOX+panitumumab was continued, and the combination therapy was stably effective. In addition, side effects were also settled, and the amount of cfDNA in plasma also decreased. An efficacy state of the patient retained SD (disease condition stabilization) although there was metastasis. These results show that it can be presumed that the amount of cfDNA in a body fluid of a patient who causes other disease complications such as pneumonia due to side effects of chemotherapy tends to increase. In addition, it was shown that a doctor can also measure the timing of drug withdrawal by tracking the amount of cfDNA.

### Example 3

The amount of DNA (cfDNA amount) in serum was measured over time for 8 colorectal cancer patients who had a recurrence after the surgical resection of the primary tumor, and the state of the tumor was observed by CT. With respect to these patients, mutation with nonsynonymous amino acid substitution was investigated for KRAS contained in the primary tumor, metastatic tumor, and the serum previously prepared from a blood sampled collect after the primary tumor was confirmed in advance.

Clinical Sample

Before or after surgical resection surgery of the primary tumor, 6 to 9 mL of peripheral blood of the patient with recurrent colorectal cancer was sampled and then centrifuged (3,000 rpm, 10 minutes), and therefore serum components were obtained. In addition, formalin-fixed paraffin-embedded (FFPE) sections of primary tumors and metastatic tumors of some patients were also used as experimental samples.

Isolation and Purification of Cell Free (cf) DNA from Serum

Isolation and purification of cfDNA from serum was carried out using QIAamp Circulating Nucleic Acid Kit (QIAGEN). The amount of serum sample provided in this kit varied depending on patients and was 2 mL to 4 ml. The isolation and purification steps of DNA were performed in accordance with the instructions attached to the kit. Final elution from a spin column was performed using 50 μL of a TE buffer.

Isolation and Purification of DNA from FFPE Section

Isolation and purification of DNA from a FFPE section was performed using QIAamp DNA FFPE Tissue Kit (QIAGEN). For each sample, three FFPE sections sliced to 10 μm were used. The isolation and purification steps of DNA were performed in accordance with the instructions attached to the kit. Final elution from a spin column was performed using 100 μL of a TE buffer.

Quantitative Determination of DNA

Quantitative determination of DNA isolated and purified from cfDNA and FFPE sections was performed using Quant-iT (registered trademark) PicoGreen (registered trademark) dsDNA Reagent and Kits (Invitrogen, Ltd). For all samples to be measured, the isolated DNA was diluted 20 times with a TE buffer and used. SAFIRA (TECAN) was used as a fluorescence measuring device. The results are shown in Table 2.

Direct Sequencing

Analysis of KRAS base sequence in surgical specimens of primary tumors and metastatic tumors, and in serum collected before or after surgical resection surgery of the primary tumors, was performed by direct sequencing. More specifically, using a primer specific to the base sequence of KRAS, cycle sequencing by BigDye Terminator method was carried out in a usual manner. The results are shown in Table 2.

Measurement of CA-19-9 in Serum

CA-19-9 in the serum was measured by the CLEIA method (chemiluminescence enzyme immunoassay method). The measurement results are shown in Table 2.

Administration of Anticancer Agent

Cetuximab and bevacizumab were administered to a case where KRAS gene mutation was not found in FFPE tissue, and to a patient in whom the KRAS gene mutation was observed therein, respectively.

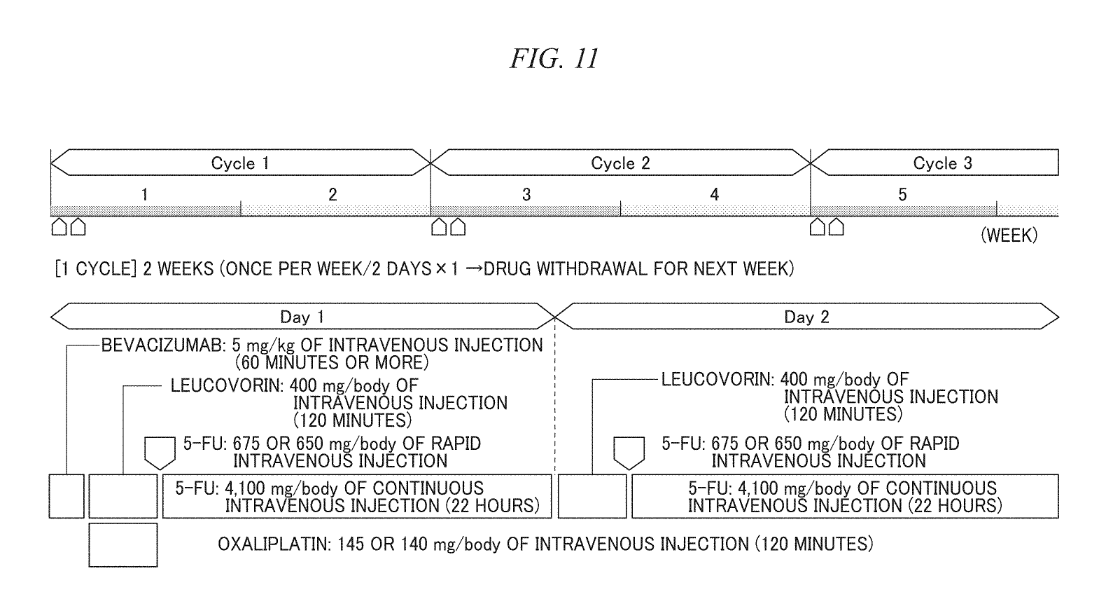

A dosage regimen in a case where bevacizumab was administered is shown in FIG. 11.

Specifically, chemotherapy of bevacizumab, an anti-VEGF inhibitor, and FOLFOX (fluorouracil/folinic acid/oxaliplatin) was performed for a patient whose primary tumor was KRAS mutant. 5 mg/kg of bevacizumab was administered by an intravenous drip injection at an administration rate of 0.5 mg/kg/min (10 minutes for 5 mg/kg) by using a 2-week interval administration method. The administration time was 90 minutes for the first time, and according to tolerability, the administration was performed for 60 minutes for the second time and 30 minutes for the third time and thereafter. For FOLFOX chemotherapy, 400 mg/body of folinic acid (leucovorin) and 145 or 140 mg/body of oxaliplatin were administered by an intravenous drip injection over 2 hours. Thereafter, fluorouracil (5-FU) was intravenously administered by a rapid intravenous drip injection at 675 or 650 mg/body, followed by a continuous intravenous drip injection of 4,100 mg/body for 22 hours.

Chemotherapy by cetuximab or panitumumab with FOLFOX (fluorouracil/folinic acid/oxaliplatin) or FORFIRI (fluorouracil/folinic acid/irinotecan) was performed for patients whose primary tumor was KRAS wild type. 800 mg/body of cetuximab was administered by an intravenous drip injection over 1 hour using a 2-week interval administration method. For FOLFOX chemotherapy, 350 mg/body of folinic acid (leucovorin) and 140 mg body of oxaliplatin were administered by an intravenous drip injection over 2 hours. Thereafter, 650 mg/body of fluorouracil (5-FU) was intravenously administered by a rapid intravenous drip injection, followed by a continuous intravenous drip injection of 4,110 mg/body for 22 hours.

Therapeutic Effect

For each patient, from the CT imaging result of the colon, the therapeutic effect was evaluated into three stages of PD (progressibility), PR (partial remission), and CR (complete remission). The evaluation results are shown in Table 2. The term “Resection” in the column of Remarks of Table 2 means that the resection surgery of the tumor tissue was performed.

TABLE 2

| | | | | cfDNA | | | Reference | |
|---|---|---|---|---|---|---|---|---|
| | | KRAS | | amount in | | | value of | |
| | Date of blood | Surgical | | serum | Therapeutic | | CA19-9: | |
| Patient | sampling | specimens | Serum | (ng/mL) | effect | Stage | 37 U/mL | Remarks |
| Case 1 | 2012 Aug. 16 | wild | Q61H | 12.2 | PD | 4 | 2.0 | |
| | 2012 Nov. 20 | wild | Q61H | 1009.1 | | | 2.0 | |
| Case 4 | 2012 Aug. 16 | G12A | G12A | 26.3 | PD | 4 | 85.2 | |
| | 2012 Dec. 12 | G12A | wild | 30.2 | | | 29.5 | Resection |
| | 2013 May 10 | G12A | G12A | 46.5 | | | 29.1 | |
| | 2013 Jun. 27 | G12A | G12A | 87.3 | | | 32.6 | |
| Case 5 | 2012 Sep. 5 | G12V | wild | 20.8 | PR | 4 | 16.5 | |
| | 2013 Mar. 8 | G12V | wild | 37.8 | | | 30.1 | |
| | 2013 Nov. 27 | G12V | wild | 5.7 | | | 22.9 | |
| Case 6 | 2012 Mar. 1 | G12D | G12D | 502.5 | PD | 4 | 3726.0 | |
| | 2013 Mar. 8 | G12D | wild | 25.8 | | | 7.7 | Resection |
| Case 7 | 2013 Jun. 27 | wild | wild | 75.0 | PR | 4 | 8.4 | |
| | 2013 Aug. 29 | wild | Q61H | 2.6 | | | 10.1 | |
| | 2013 Nov. 27 | wild | wild | 9.0 | | | 8.3 | |
| Case 8 | 2012 Dec. 12 | wild | wild | 20.7 | PD | 4 | 169.8 | |
| | 2013 Jul. 3 | wild | wild | 74.4 | | | 10.9 | |
| Case 10 | 2013 Jul. 4 | wild | wild | 237.0 | PR | 4 | 2.0 | |
| | 2013 Nov. 27 | wild | wild | 10.4 | | | 2.0 | |
| Case 13 | 2012 Aug. 23 | wild | wild | 88.1 | CR | 4 | 11.9 | |
| | 2013 Aug. 8 | wild | wild | 13.9 | | | 25.1 | |

More specifically, a patient of Case 7 started chemotherapy with cetuximab and FOLFOX on Jan. 31, 2013 and was PR until Apr. 16, 2015. In other words, in the patient of Case 7, anticancer agent therapy was effective for a long period of about 1 year and 8 months from Aug. 29, 2013 on which a decrease in the cfDNA amount was confirmed, which is about 2 years from the start of the anticancer agent therapy.

In addition, a patient of Case 10 started chemotherapy with cetuximab and FOLFOX on Jul. 15, 2013, and was PR until Feb. 5, 2015. In other words, in the patient of Case 10, anticancer agent therapy was effective for a long period of about 1 year and 2 months from Nov. 27, 2013 on which a decrease in the cfDNA amount was confirmed, which is about 1 year and 6 months from the start of the anticancer agent therapy.

A patient of Case 13 started chemotherapy with cetuximab and FOLFOX on Jun. 19, 2012, and was PR until at least 16 May 2014, but recurrence was confirmed on Aug. 6, 2014. In other words, in the patient of Case 13, anticancer agent therapy was effective for a long period of about 9 months from Aug. 8, 2013 on which a decrease in the cfDNA amount was confirmed, which is about 2 years from the start of the anticancer agent therapy.

A patient of Case 5 received chemotherapy with bevacizumab and FOLFOX from Aug. 22, 2012 to September 2 of the same year, from Feb. 20, 2013 to March 3 of the same year, and from Oct. 30, 2013 to November 10 of the same year. It was confirmed that the patient was PR at Sep. 2, 2012 and confirmed that the patient was PR until Dec. 20, 2013.

As a result, in the cancer patients (Cases 5, 7, 10, and 13) whose therapeutic effect was PR or CR and efficacy of the anticancer agent was confirmed, the average value of the amount of cfDNA in serum on the final day of sampling blood was 9.8 ng/mL, a standard deviation was 3.40 ng/mL, and variation was small. In other words, it was found that the amount of cfDNA in blood was very small at least in the effective case. On the other hand, as a result for the cancer patients (Cases 1, 4, 6, and 8) whose therapeutic effect was PD and efficacy of the anticancer agent was not shown, the average value of the amount of cfDNA in serum on the final day of sampling blood exceeded 100 ng/mL, and variation was large. As an actual criteria, an average value+3σ in the effective case is appropriate because the value thereof is equal to or less than the DNA amount of a minimum value in the non-effective case. That is, as a criteria, the reference value of the cfDNA amount in serum could be set to 20 ng/mL as an indicator showing that therapy of the EGFR inhibitor or the VEGF inhibitor was effective.

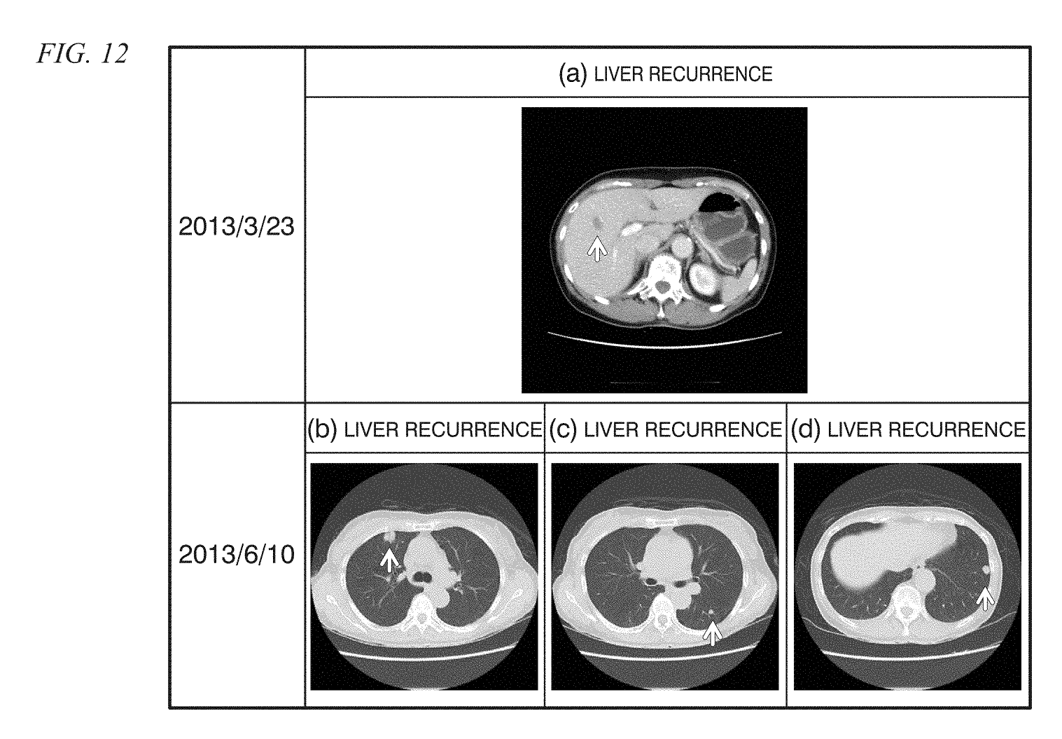

In addition, as a result, KRAS gene mutation (G12A) which was to the same as the primary tumor was observed from cfDNA before the resection surgery of the primary tumor of cancer patient in Case 4 which was not effective. However, G12A was not detected from cfDNA after the resection surgery of the primary tumor. However, after the surgery, G12A was detected from serum KRAS and recurrence occurred after 4 months, and therefore bevacizumab and FOLFIRI were not effective. The amount of cfDNA obtained by monitoring the cancer patient in Case 4 was 87.3 ng/mL which was a relatively high concentration. FIG. 12 is a figure of CT imaging of a cancer patient in Case 4.

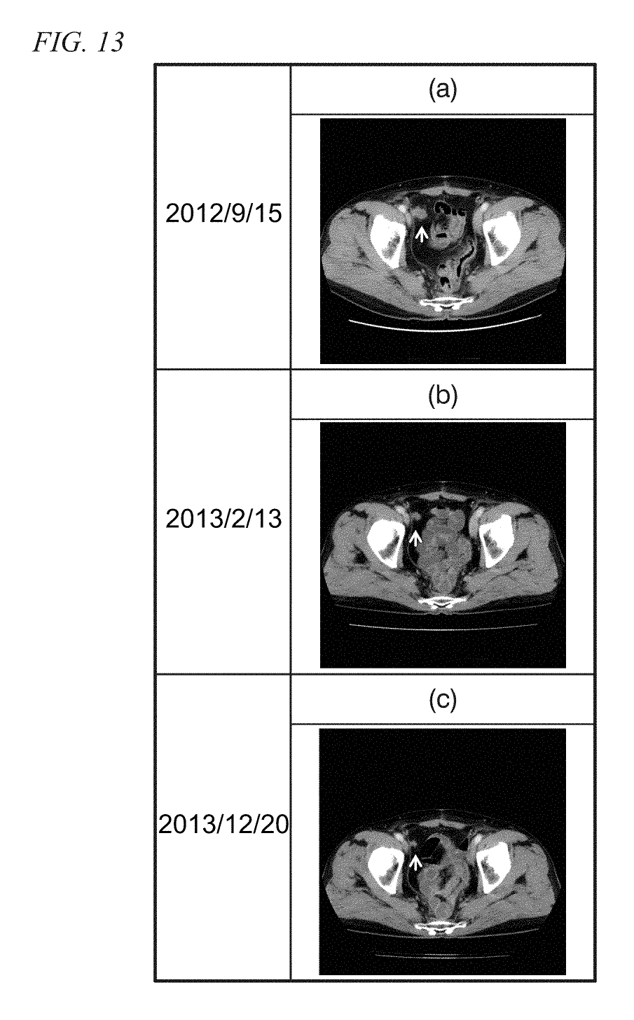

The cancer patient in Case 5 was a case in which the combination therapy of bevacizumab+FOLFOX was prominently effective in the long term. The patient maintained a state in which a tumor reduction rate (reduction effect) did not change for half a year or more. The tumor reduction rate was calculated from a diameter of the tumor in the CT image, and in a case where the tumor was present in multiple sites, the tumor reduction rate was calculated by summing diameters thereof. FIG. 13 is a CT imaging diagram of a cancer patient in Case 5. In the figure, arrows indicate tumor recurrence sites. The amount of cfDNA in serum of the patient at the final day of sampling blood was 5.7 ng/mL.

In the simple prediction of the efficacy of the therapy including the EGFR inhibitor or the VEGF inhibitor in the patients with recurrent colorectal cancer in the examples, quantitatively determining the amount of DNA circulating in peripheral blood, particularly serum or plasma, is useful in view of need of only a small amount of a blood sample and being a simple test method. In addition, by tracking the variation of the amounts of cfDNA, a doctor can check whether the therapy works properly. Furthermore, it was possible to accurately confirm whether the therapy was working properly in a similar manner as an existing biomarker such as CA19-9, which is a common tumor marker.

What is claimed is:

1. A method for evaluating a health condition, comprising:
measuring a test amount of cell free DNA per unit amount of a body fluid sample collected from a subject after tumor resection; and
comparing the measured test amount of cell free DNA with a reference value so as to evaluate a health condition of the subject: wherein
the reference value is any one of a predetermined threshold value; an amount of cell free DNA per unit amount of a body fluid sample collected from the subject before the tumor resection; an amount of cell free DNA per unit amount of a body fluid sample collected from the subject between 1 week and 3 months after the tumor resection; and an amount of cell free DNA per unit amount of a body fluid sample collected from the subject by the time of collecting the body fluid sample for measuring the test amount of cell free DNA, and
when evaluating the health condition of the subject, in a case where the test amount of cell free DNA is higher than the reference value, it is evaluated that a tumor of the subject has increased or new metastasis has occurred or there is a high possibility of an increase of a tumor or of occurrence of new metastasis, or that while a tumor of the subject has not increased and no new metastasis has occurred, the health condition has deteriorated or there is a high possibility of deterioration of the health condition.

2. The method for evaluating a health condition according to claim 1, wherein
the body fluid sample is a body fluid sample collected from the subject after the tumor resection over time, and
the test amount of cell free DNA per unit amount of the body fluid sample is monitored so as to evaluate the health condition of the subject over time.

3. The method for evaluating a health condition according to claim 2, wherein
in a case where the test amount of cell free DNA per unit amount of the body fluid sample of the subject is increasing, it is evaluated that a tumor of the subject has increased or new metastasis has occurred or there is a high possibility of an increase of a tumor or occurrence of new metastasis, or while a tumor of the subject has not increased and no new metastasis has occurred, the health condition has deteriorated or there is a high possibility of deterioration of the health condition.

4. The method for evaluating a health condition according to claim 1, wherein
the subject has received chemotherapy, and
when evaluating the health condition of the subject, in a case where the test amount of cell free DNA is higher than the reference value, it is evaluated that a side effect due to the chemotherapy has occurred or there is a high possibility of occurrence of the side effect due to the chemotherapy.

5. The method for evaluating a health condition according to claim 1, wherein
the subject has received chemotherapy, and
when evaluating the health condition of the subject, in a case where the test amount of cell free DNA is higher than the reference value, it is evaluated that the chemotherapy is ineffective.

6. The method for evaluating a health condition according to claim 4, wherein
a chemotherapeutic agent used for the chemotherapy is one or more selected from the group consisting of fluorouracil, leucovorin, oxaliplatin, capecitabine, tegafur/gimeracil/oteracil potassium, irinotecan, bevacizumab, cetuximab, and panitumumab.

7. The method for evaluating a health condition according to claim 1, wherein
the reference value is an amount of cell free DNA per unit amount of the body fluid sample collected from the subject when 42 to 90 days have passed from the tumor resection.

8. The method for evaluating a health condition according to claim 1, wherein
the body fluid sample is selected from the group consisting of blood, serum, plasma, urine, saliva, semen, thoracic exudate, cerebrospinal fluid, tears, sputum, mucus, lymph fluid, cytosol, ascites, pleural effusion, amniotic fluid, bladder lavage solution, and bronchoalveolar lavage solution.

9. The method for evaluating a health condition according to claim 1, wherein
the body fluid sample is serum or plasma.

10. The method for evaluating a health condition according to claim 9, wherein
the reference value is 1000 ng of an amount of cell free DNA per mL of plasma.

11. The method for evaluating a health condition according to claim 1, wherein
the amount of cell free DNA per unit amount of the body fluid sample is measured by an absorbance method, an intercalation method, a real-time PCR method, a digital PCR method, a next generation sequencing method, or an electrochemical detection method.

12. The method for evaluating a health condition according to claim 1, wherein
the tumor is one of the group consisting of metastatic medulloblastoma, gastrointestinal stromal tumor, dermatofibrosarcoma protuberans, colon rectum cancer, colorectal cancer, lung cancer, non-small-cell lung cancer, small cell lung cancer, chronic myeloproliferative disease, acute myelogenous leukemia, thyroid cancer, pancreatic cancer, bladder cancer, kidney cancer, melanoma, breast cancer, prostate cancer, ovarian cancer, cervical cancer, head and neck cancer, brain tumor, hepatocellular carcinoma, hematologic malignancy, and pre-cancer causing these cancers.

13. The method for evaluating a health condition according to claim 1, wherein
the evaluated health condition of the subject is used as a material for determining whether to continue or stop the chemotherapy or to change the chemotherapeutic agent to be used.

14. A method for predicting long-term efficacy of an anticancer agent, comprising:
measuring an amount of cell free DNA per unit amount of a body fluid sample collected from a subject through two or more collections over time so as to monitor the amount of cell free DNA per unit amount of the body fluid sample of the subject; and
comparing the obtained amount of cell free DNA with a predetermined reference value; wherein
it is predicted that long-term efficacy of an anticancer agent is obtained for the subject in a case where the amount of cell free DNA decreases from a value greater than the reference value to the reference value or less, or in a case where the amount of cell free DNA of each body fluid sample obtained through all of the collections is equal to or less than the reference value; and
it is predicted that long-term efficacy of the anticancer agent is not obtained for the subject in a case where the amount of cell free DNA increases from a state equal to or less than the reference value to a value greater than the reference value, or in a case where the amount of cell free DNA of each body fluid sample obtained through all of the collections is greater than the reference value.

15. The method for predicting long-term efficacy of an anticancer agent according to claim 14, wherein
the body fluid sample is serum or plasma.

16. The method for predicting long-term efficacy of an anticancer agent according to claim 15, wherein
the reference value is 20 ng of the amount of cell free DNA per mL of serum or plasma.

17. The method for predicting long-term efficacy of an anticancer agent according to claim 14, wherein
the long-term efficacy means that a volume increase, metastasis, or recurrence of tumor tissue does not occur for at least 6 months.

18. The method for predicting long-term efficacy of an anticancer agent according to claim 14, wherein
the subject suffers from one or two or more selected from the group consisting of colorectal cancer, colon cancer, rectal cancer, lung cancer, liver cancer, breast cancer, ovarian cancer, prostate cancer, renal cancer, esophageal cancer, head and neck cancer, uterine cancer, and cervical cancer.

19. The method for predicting long-term efficacy of an anticancer agent according to claim 14, wherein
the anticancer agent is one or more selected from the group consisting of an EGFR inhibitor and a VEGF inhibitor.
